(19) United States
(12) Patent Application Publication
(10) Pub. No.: US 2023/0295546 A1
(43) Pub. Date: Sep. 21, 2023

(54) APPARATUS FOR GROWING BIOMASS

(71) Applicant: NBTECH AB, Järfälla (SE)

(72) Inventor: Nathalie BEREZINA, Järfälla (SE)

(21) Appl. No.: 18/017,080

(22) PCT Filed: Jul. 8, 2021
(86) PCT No.: PCT/EP2021/068973

§ 371 Date: Jan. 19, 2023

(30) Foreign Application Priority Data
Jul. 27, 2020 (EP) 20187902.0

(51) Int. Cl. C12M 1/16; C12M 1/12; C12M 3/00; C12M 1/26; C12M 1/00

(52) U.S. Cl. CPC C12M 21/16; C12M 23/04; C12M 23/48; C12M 23/50; C12M 33/14

(57) ABSTRACT

An apparatus (100, 700, 8001, 802, 803) for growing biomass, wherein the apparatus comprises: at least one plate (110, 210, 310, 410, 710) comprising at least two plate sections (120, 220, 320, 331, 332, 333, 420, 421, 422) configured to be movable between an opened position and a closed position. In the closed position the at least two plate sections jointly form a first surface for receiving and holding a growth medium for growing biomass and in the opened position the at least two plate sections are pivoted away from the closed position such that the growth medium is slidably released from the at least one plate. The at least one plate is movably supported on at least one first railing (150, 750), wherein the apparatus further comprises a first drive mechanism (715) for independently moving each of the at least one plate along the at least one first railing; and the at least two plate sections are pivotable about a first axis (251) and a second axis (252) respectively.

### TECHNICAL FIELD

The inventive concept described herein generally relates to the culture of heterogeneous biomass. More specifically, the present inventive concept relates to an apparatus for growing heterogeneous biomass in a controllable environment.

### BACKGROUND

The use of biomass for the production of energy has attracted and continues to attract attention and interest in the current global environmental situation. The growing desire of industries to move away from fossil fuels and into renewable energy has increased the priority given to the production of biomass from organic matter derived from vegetable organisms, animals, bacteria and fungi, amongst others.

One of the most commonly seen approach to the production of biomass is represented by processes for growing heterogeneous biomass, for example through solid state fermentation techniques or cultivation of fungi and insect production.

However, growing of heterogeneous biomass suffers from several issues such as difficulty of biomass control, heterogeneity among individuals and contamination of growing areas and of the biomass itself. Solutions attempting to resolve these issues have been proposed in the past but remain slightly insufficient. For example, for solid state fermentation techniques, specifically designed reactors have been used to ensure the control and the sterility of the biomass growing process, but such solution has been found to limit the volume and amount of produced biomass making it only suitable for the production of biomass with high added value molecules. For the cultivation of fungi and insect production, the currently privileged approach is the accumulation of rearing in breeding units requiring transportation of these breeding to operation areas e.g. as disclosed in prior art WO2014/171829. The main problem of using such approach lies in the use devices e.g. stack of trays or containers, inadequate for enabling the complete growing process of the biomass therefore requiring a plurality of different devices and their manipulation and transport through various areas increasing the risk of contamination of the biomass.

Hence, it is an object of the present invention to try and overcome at least some of the deficiencies of present equipment used for growing biomass specifically pertaining to the capacity a device for growing biomass to individually enable the complete growing process of biomass and to the mitigation of the contamination risk.

### SUMMARY OF THE INVENTION

It is an object of the present inventive concept to mitigate, alleviate or eliminate one or more of the above-identified deficiencies in the art and disadvantages singly or in combination. According to a first aspect of the inventive concept, these and other objects are achieved in full, or at least in part, by an apparatus for growing biomass, wherein the apparatus comprises at least one plate comprising at least two plate sections configured to be movable between an opened and a closed position, wherein in the closed position the at least two plate sections jointly form a first surface for receiving and holding a growth medium for growing biomass and wherein in the opened position the at least two plate sections are pivoted away from the closed position such that the growth medium is slidably released from the at least one plate. The at least one plate is movably supported on at least one first railing, wherein the apparatus further comprises a first drive mechanism for independently moving each of the at least one plate along the at least one first railing and wherein the at least two plate sections are pivotable about a first axis and a second axis respectively.

The apparatus may further comprise at least one grid comprising at least two grid sections movable between an opened position and a closed position, wherein in the closed position the at least two grid sections jointly form a second surface and wherein said at least two grid sections comprise a meshing having a plurality of mesh openings for receiving and separating the growth medium. Moreover, in the opened position the at least two grid sections are pivoted about a third and a fourth axis respectively and away from the closed position such that separated growth medium falls off the at least one grid, wherein the at least one grid is movably supported on at least one second railing and wherein the at least one first railing is positioned above the at least one second railing such that the at least one plate and the at least one grid are movable to a discharging position in which the at least one plate and the at least one grid are superimposed. The apparatus further comprises a second drive mechanism for independently moving each of the at least one grid along the at least one second railing.

In use, the plate section(s) are moved to their closed position thereby forming a surface for receiving a growth medium. The growth medium then enables biomass to grow on each plate. By movably supporting each plate on the first railing, individual movement of each plate is enabled, in turn enabling each respective plate to be moved away and harvested as needed depending on for example varying growing rapidity of the biomass on the different plates. It will be appreciated that each of the at least one plate is movable independently from one another on the at least one first railing and that each of the at least one plate is independently supported on the at least one first railing. It will further be appreciated that each of the at least one plate of the apparatus may be movably suspended off the at least one first railing i.e. having the at least one first railing positioned above the at least one plate. Since the first railing and the second railing hold the plates and the grids on different heights, the plates and the girds are movable to the discharging position in which a plate to be emptied is positioned superimposed above a grid. Once above the grid, the growth medium on the plate can be moved to the grid by moving the plate to its opened configuration. The grid then separates the grown biomass from the growth medium and undesirable substances by preventing larger particles, i.e. non-harvestable biomass, from moving through the plurality of mesh openings of the meshing of the grid and letting smaller particles, i.e. harvestable biomass, through the plurality of mesh openings of the meshing of the grid. The first and second drive mechanisms enable movement of the plates and grids between their different positions for growing biomass, harvesting grown biomass and decontaminating the plate and the grid. It will be appreciated that the apparatus for growing biomass comprising the at least one plate and the device for separating a growth medium comprising the at least one grid are interrelated in that their movement on respective railings and their respective function enable successful growing of biomass. In other words, the apparatus permits the biomass to grow on a growth medium deposited on the at least one plate and the device permits grown biomass to be separated from the growth medium and other undesirable substances thereon by the at least one grid once the biomass is successfully grown and released from the at least one plate onto the at least one grid. The interrelated functionality of the at least one plate and the at least one grid thus enables the apparatus for growing biomass and the device for separating the growth medium to grow biomass and harvest grown biomass cyclically. Since the present invention is aimed at the production of high volumes of biomass, the apparatus for growing biomass and the device for separating growth medium are closely interrelated.

Such an apparatus for growing biomass thus enables a highly efficient and complete process for growing biomass since the plates and grids are individually operated for growing and harvesting using only little space. The individual operation of each plates and grids therefore permits the apparatus to conduct each parts of the biomass growing process without requiring additional equipment or transport of the apparatus through different areas or spaces.

The present invention is hereby advantageous in that in the closed position the first surface formed by the at least two plate sections of the at least one plate enables a flat surface for the growth medium to grow biomass resulting in great distribution of the growth medium over the area of said first surface reducing the risks of uneven accumulation of growth medium on different parts of the area which would generate uneven growth of the biomass on the at least one plate. Moreover, in the opened position, the at least two sections of the at least one plate may form an angle with a horizontal plane formed by the first surface in the closed position. The angle of the at least two sections with the horizontal plane enables a smooth discharge of the growth medium and its growing biomass by gravitational effect without damaging it. It will be appreciated that the at least one plate is formed of a metallic material and/or a plastic material enabling the growth medium to slide off the at least two sections when the at least one plate is in the opened position with minimum friction between the plate and the growth medium. The present invention is further advantageous in that at least one of the at least two plate sections may be extendable such that the area of the surface of the at least one of the at least two plate sections may be increased, resulting in a larger area of the first surface formed by the at least two plate sections of the at least one plate when in the closed position. By the term “extendable” it is here meant that at least one dimension (width and/or length if the two plate sections are of rectangular or square shape) of at least one of the at least two plate sections may be varied to increase the area of the at least one of the at least two plate sections, for example via a telescopic mechanism. The possibility of increasing the area of the first surface provides the advantage of adapting the area of the first surface of the at least one plate to the variation of the density of the growing biomass. In other words, for a plate on which the a larger density of biomass is observed on the growth medium, at least one of the at least two plate sections may be extended such that the total area of the first surface accommodates the biomass without compromising its growth.

The present invention further permits the at least one first railing to encompass a plurality of configurations for supporting the at least one plate. For example, the at least one first railing may comprise two railings supporting the at least one plate on two sides of its perimeter or the at least first railing may comprise only one railing supporting the at least one plate in its middle. It will be appreciated that the at least one plate preferably embodies a rectangular or square shape ensuring greater support of the plate on the at least one railing, but the at least one plate may also embody other dimensional shapes provided the capacity of being securely supported by the at least one railing. It will additionally be appreciated the at least one first railing and second railing may comprise curvatures or angles in their elongation, allowing the trajectory of the at least one plate and the at least one grid to be adaptable to the growing space in which they are enclosed.

The present invention is further advantageous in that the first and second axis may elongate along any edges of the perimeter of each of the at least one plate sections permitting each plate section to pivot between the closed and opened positions enabling the growth medium to be discharged from the at least one plate. The first and second axis may also elongate along the middle of each of the at least two plate sections or along their respective diagonals. The different elongations or positioning of the first and second axis is therefore advantageous in that is enables the at least one plate to embody a variety of different pivoting configurations.

The present invention is further advantageous in that at least one grid of the apparatus comprises the same advantages as the at least on plate with regards to its movement, support on the at least one second railing and transition between the opened and the closed position. It will furthermore be appreciated that the at least one grid embodies dimensions and shape similar to the dimensions and shape of the at least one plate to ensure efficient and secure transfer of the growth medium between the two and eradicate the risk of biomass loss or damaging during said transfer. The at least one grid is further advantageous in that the meshing and its mesh openings enable the separation of the grown biomass from the rest of the growth medium and from the undesirable substances comprised thereon. It will further be appreciated that the second drive mechanism enhances the separation function of the at least one grid by enabling movements of the at least one grid along the at least one second railing on which it is supported. It is further envisioned that the at least one grid comprises a vibration mechanism enabling vibration of the at least one grid in any direction, e.g. upward motion, downward motion, circular motions, lateral motion, etc. to enhance the separation effect of its meshing.

The present invention is further advantageous in that the positioning of the at least one first railing above the at least one second railing enables the alignment and the superimposition of the at least one plate with the at least one grid resulting again in efficient and secure transfer of the growth medium between the two and eradicates the risk of biomass loss or damaging during said transfer.

By the term “growth medium” it is here meant a substrate or growing support material enabling ideal conditions and characteristics for biomass to grow thereon successfully. A growing medium may be represented, but not limited to soil, coconut coir, growstones, expanded clay aggregate, perlite etc. A growing substrate may be a combination or mixture of a plurality of substrates including, but not limited to, water, vitamins, oligo-elements, etc.

By the term “meshing” and “mesh openings” it is here meant an arrangement of interlocked material (metal, plastic, etc.) links enabling a sieving or filtering function. The mesh openings of the meshing comprise specific dimensions allowing only particles of a specific granulometric dimensions to pass therethrough and retain the particles having a greater size.

According to an embodiment of the present invention, the at least one plate and the at least one grid further comprise borders arranged along at least a portion of a perimeter of the first surface and along at least a portion of a perimeter of the second surface respectively. The present embodiment is advantageous in that the borders prevents the growth medium and the biomass growing thereon from falling off the first surface during the growth process of the biomass e.g. during movement of the at least one plate or the at least one grid therefore ensuring a higher volume of biomass produced and mitigating the risk of contamination of the other plates and grids positioned nearby. Similarly, the present embodiment prevents the growth medium and grown biomass from falling off the at least one grid specifically during separation. The present embodiment is further advantageous in that the borders increase the retention of water and/or nutrient on the first surface and second surface in a case wherein the growth process is complemented by operations of addition of water and/or nutrient, resulting in a higher quality of the biomass grown. Additionally, it will be appreciated that the borders preferably extend in an upward direction perpendicular to said first surface and second surface respectively.

According to an embodiment of the present invention, the first axis and the second axis may be co-linear. Furthermore, the third and the fourth axis may be co-linear. The present embodiment is advantageous in that it enables a greater variety of pivoting configurations of the at least one plate and the at least one grid resulting in a better adaptability to various spaces in which the apparatus is used. For example, the co-linearity of the first and second axis and the co-linearity of the third and fourth axis render possible a configuration of the apparatus in which the at least two plate sections and/or the at least two grid sections are movably supported by only one first railing and one second railing positioned in their respective centers.

According to an embodiment of the present invention, one or more of said at least one plate may further comprise a respective thermoregulating device arranged along at least a portion of the perimeter of the first surface, wherein the thermoregulating device may be one of a refrigerant conduit, a cooling coil, a water pipe, and a heating coil. The present embodiment is advantageous in that it permits the regulation and control of the temperature of the at least two plate sections on which the growth medium is held during the biomass growing process. The thermoregulating device enables the heating and cooling of the first surface of the at least one plate according to parameters of temperature best suited for the type of biomass grown. The present embodiment is therefore advantageous in that it increases the quality of the biomass grown and ultimately results in a more efficient biomass growing process.

According to an embodiment of the present invention, the apparatus may comprise a plurality of said at least one grid wherein the grids are positioned elevated from one another to form part of a multi-layered grid assembly. By the term “elevated” it is here meant vertically higher i.e. providing the possibility of being superimposed if aligned. The present embodiment is advantageous in that it allows a plurality of at least one grids to be superimposed at the discharging position thus provide a plurality of meshing to be solicitated for performing the separation of the grown biomass from the growth medium and undesirable substances.

According to an embodiment of the present invention, each of the grids of the multi-layered grid assembly may be provided with differently sized mesh openings. The present embodiment is advantageous in that it permits several sizes of particles to be separated. In other words, the present embodiment enables particles of grown biomass and/or undesirable substances of various granulometric dimensions to be separated when passing through the different grids of the multi-layered grid assembly. The present embodiment is therefore advantageous in that, by providing a plurality of grids, each having differently sized mesh openings, a gradual separation of matter is enabled, such that different types of particles can be collected/harvested from each grid. Larger particles are retained on grids positioned further up in the multi-layer grid assembly and smaller particles pass through to lower positioned grids. The grids can then be emptied one-by-one in, e.g. a separate container or tray, to collect respective particle types.

According to an embodiment of the present invention, the at least one second railing may comprise a separate channel for support of each grid of the multi-layered grid assembly, and wherein the second drive mechanism is configured to move each grid independently along the at least one second railing. The present embodiment is advantageous in that it enhances the efficiency to the separation function of each grids through their respective meshing by enabling movement of each grids along the at least one second railing. Additionally, the separate channel provides one or more support surface for supporting the at least one grid. Furthermore, the provision of a separate channel for each grid enables independent movement of said grids along the second railing permitting the removal of one or more of the grids to adapt the separation of the growth medium to specific granulometric dimensions of particles. In other words, the present embodiment permits the adjustability of the separation function of the multi-layered grid assembly to the type of growth medium and grown biomass being separated. The present embodiment is further advantageous in that the second drive mechanism enables control of the movement of the grids of the multi-layered grid assembly in the separate channel in turn permitting the rapidity of the separation to be adapted to the fragility of the biomass grown.

According to an embodiment of the present invention, the multi-layered grid assembly may comprise at its bottom a container comprising at least two container sections movable between an opened position and a closed position, wherein in the closed position the at least two container sections may jointly form a third surface receiving and holding the separated growth medium, wherein in the opened position the at least two container sections may be pivoted away from the closed position such that separated growth medium is slidably released from the container. By providing the multi-layer grid assembly with a container at its bottom, any separated particles or sorted matter passing the lowest-positioned grid of the multi-layered grid assembly falls into the container adapted to contain the separated particles or sorted matter and subsequently release them on e.g. a transport mechanism for transport to an area for further transformation of the biomass or for transport to a dispensing area for separated particles or sorted matter that are undesirable substances.

According to an embodiment of the present invention, the first drive mechanism may comprise at least one chain adapted to engage at least one edge of the at least one plate, and wherein the first drive mechanism may further comprise motor driven sprockets for moving the at least one chain back and forth along the at least one first railing. Moreover, the second drive mechanism may comprise at least one chain adapted to engage at least one edge of the at least one grid, wherein the second drive mechanism comprises motor driven sprockets for moving the at least one chain back and forth along the at least one second railing. The present embodiment is advantageous in that it enables the at least one chain of the first and second drive mechanism to engage the at least one plate and the at least one grid respectively and to force the at least one plate and the at least one grid back and forth along the at least one first railing and the at least one second railing respectively. The present embodiment further allows the motor driven sprockets to engage the chains and power the chains for movement of the at least one plate and at least one grid. Additionally, for a multi-layered grid assembly, the second drive mechanism enables each grid to be moved individually by providing respective chains and motor driven sprockets for each grid. It will be appreciated that the first and second drive mechanisms allow the movement of the at least one plate and at least one grid to be fully automated.

According to an embodiment of the present invention, the at least one plate may be attached to the at least one first railing via at least one third railing oriented perpendicular to the at least one first railing such that the at least one plate and the at least one third railing may be movable together along the at least one first railing, wherein the at least one plate may be movably supported on the at least one third railing for movement along the at least one third railing, and wherein the apparatus may further comprise a third drive mechanism for moving the at least one plate along the at least one third railing. Furthermore, the at least one grid may be attached to the at least one second railing via at least one fourth railing oriented perpendicular to the at least one second railing such that the at least one grid and the at least one fourth railing may be movable along the at least one second railing, wherein the at least one grid may be movably supported on the at least one fourth railing for movement along the at least one fourth railing, and wherein the apparatus may further comprise a fourth drive mechanism for moving the at least one grid along the at least one fourth railing. The provision of the at least one third railing perpendicular to the at least one first railing and the provision of the at least one fourth railing perpendicular to the at least one second railing adds another degree of freedom of movement of each plate and each grid thus enabling a greater flexibility of how the plates and the grids are positioned and moved within an area during growing, harvesting and decontamination of the plates and grids. The present embodiment is further advantageous in that it provides many possible positions of the at least one plate and the at least one grid through the use of different length configurations for each of the railings. The present embodiment is therefore advantageous in that it permits sufficient distancing of the at least one plate and the at least one grid from the trajectory followed during growth of the biomass, i.e. along the at least one first railing and along the at least one second railing, to ensure that other operations performed on the plates and grids, e.g. decontamination, do not interfere with the biomass growing process. It will further be appreciated that the at least one third railing and the at least one fourth railing are characterized similarly as the at least one first railing and the at least one second railing pertaining their configuration for supporting the at least one plate.

According to an embodiment of the present invention, the apparatus may comprise a plurality of plates movably supported on respective first railings and movable by the first drive mechanism wherein each of the first railings may be configured to be positioned one above the other and above the at least one second railing. The present embodiment is advantageous in that the provision of multiple first railings arranged above one another is a convenient way in which to make the plates independently movable from one another over a larger operational range along the first railing(s) since the difference in height enables the plates to pass under/above other plates. Furthermore, the present embodiment enables a greater number of plates to be used for growing biomass without requiring a considerable increase in dimensions of the space in which the apparatus is used ultimately resulting in the enhancement of the production of biomass. It will be appreciated that for the configuration of the apparatus related in the present embodiment are sequentially movable to the discharging position at which they are sequentially superimposed over the at least one grid enabling unobstructed transfer of the growth medium from the plates to the grid.

A feature described in relation to one aspect may also be incorporated in other aspects, and the advantage of the feature is applicable to all aspects in which it is incorporated.

Other objectives, features and advantages of the present inventive concept will appear from the following detailed disclosure, from the attached claims as well as from the drawings.

Generally, all terms used in the claims are to be interpreted according to their ordinary meaning in the technical field, unless explicitly defined otherwise herein. Further, the use of terms “first”, “second”, and “third”, and the like, herein do not denote any order, quantity, or importance, but rather are used to distinguish one element from another. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

### BRIEF DESCRIPTION OF THE DRAWINGS

The above, as well as additional objects, features and advantages of the present inventive concept, will be better understood through the following illustrative and non-limiting detailed description of the present inventive concept, with reference to the appended drawings, wherein:

FIG. 1 schematically illustrates a top view of an apparatus for growing biomass;

FIGS. 2a-2b schematically illustrate perspective views of different configurations of a plate of an apparatus for growing biomass;

FIG. 2c schematically illustrates a perspective view of a grid of an apparatus for growing biomass;

FIGS. 3a-3d schematically illustrate side views of different pivoting configurations of the plate sections of a plate of an apparatus for growing biomass;

FIGS. 4a-4c schematically illustrate top views of different configurations of pivoting axis of the plate and grid of an apparatus for growing biomass;

FIG. 5a schematically illustrates a perspective view of a multi-layered grid assembly;

FIG. 5b schematically illustrates a side view the multi-layer grid assembly;

FIG. 6 schematically illustrates an exploded view of a multi-layer grid assembly;

FIG. 7 schematically illustrates a side view of an alternative configuration of apparatus for growing biomass;

FIG. 8 schematically illustrates a top view of an arrangement of apparatus for growing biomass.

The figures are not necessarily to scale, and generally only show parts that are necessary in order to elucidate the inventive concept, wherein other parts may be omitted or merely suggested.

### DETAILED DESCRIPTION

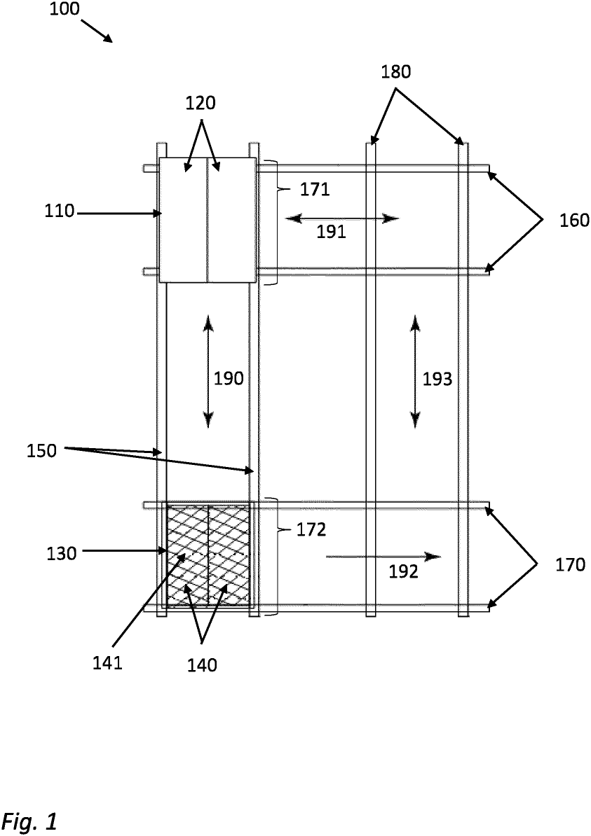

FIG. 1 illustrates an apparatus 100 for growing biomass according to a first embodiment. The apparatus 100 comprises a plate 110 movably supported on a first railing 150 and being composed of two plate sections 120. The first railing 150 is characterized by two rails supporting opposite edges of the longer dimension of the plate 110. The plate 110 is shown in FIG. 1 in a closed position enabling the two plate sections 120 to jointly form the first surface of the plate 110. FIG. 1 further shows the plate 110 positioned at a loading position 171 at which the plate 110 is ready to receive and hold a growth medium (not shown) on its first surface. FIG. 1 further depicts the movement 190 the plate 110 follows along the first railing 150 to progress to and from a discharging position 172. The movement 190 of the plate 110 is enabled by the first drive mechanism (not shown in FIG. 1 but detailed later in the description). The apparatus 100 shown in FIG. 1 further comprises a grid 130 movably supported on a second railing positioned under the first railing 150 and encompassing similar characteristics as the first railing 150 supporting the plate 110. The second railing is therefore not visible in FIG. 1 as the first railing 150 and the plate 110 supported thereon are elevated directly above the second railing. The grid 130 is further shown in FIG. 1 having two grid sections 140 jointly forming the second surface of the grid 130 and formed of a meshing 141 enabling the separation function of the grid 130. FIG. 1 depicts the grid 130 being positioned at the discharging position 172 at which the grid 130 is ready to receive the growth medium and the biomass grown thereon. The movement of the grid 130 along the second railing is in the same direction as the movement 190 of the plate 110 and is enabled by a second drive mechanism (not shown but described later in the description). It is embodied that the movement 190 of the plate 110 and the movement of the grid are independent from one another as they are enabled by different drive mechanisms. FIG. 1 further illustrates a third railing 160 formed of two rails similarly as the first railing 150 and adapted to movably support the plate 110. The third railing 160 is shown oriented perpendicularly to the first railing 150 permitting the movement 191 of the plate 110 away from the first railing 150. It is further embodied that the movement 191 of the plate 110 along the third railing 160 is enabled by the first drive mechanism. Furthermore, FIG. 1 illustrates a fourth railing 170 characterized similarly as the third railing 160 but for movably supporting the grid 130 and enabling the movement 192 of said grid 130 away from the second railing in a perpendicular direction by means of the second drive mechanism. FIG. 1 further shows a fifth railing 180 elongating in a parallel direction as the first railing 150 and enabling the movement 193 of the plate 110 along its tow rails. Although not visible in the top view illustrated in FIG. 1, The apparatus 100 further comprises a sixth railing characterized similarly as the fifth railing 180 but for movably supporting the grid 130 and enabling its movement parallel to the movement 193. It is envisioned that the connection between the first railing 150 and the third railing 160, and the connection between the third railing 160 and the fourth railing 170 do not hinder the capacity of the plate 110 to securely hold the growth medium. It is additionally embodied that the third railing 160 may be attached to the plate 110 and movable along the first railing 150 according to movement 190. For such embodied configuration, the use of the fifth railing 180 is not required. Similarly, it is additionally embodied that that the fourth railing 170 may be attached to the grid 130 and movable along the second railing. For such embodied configuration, the use of the sixth railing is not required.

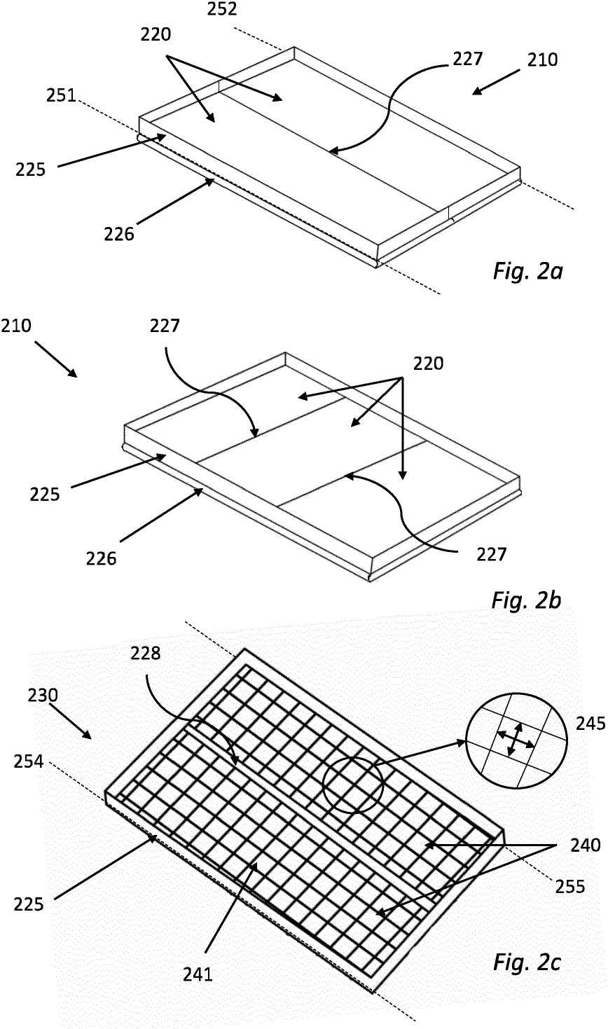

Referring now to FIG. 2a, there is shown a perspective view of a plate 210 in a closed position. FIG. 2a illustrates the plate 210 having two plate sections 220 jointly forming the first surface said plate 210. FIG. 2a further shows borders 225 arrange along the perimeter of the plate 210, more specifically along at least a portion of the perimeter of each plate sections 220. The borders 225 elongate in an upward direction perpendicular to the first surface formed by the two plate sections 220 and may preferably have a height ranging from 0.5 cm to 10 cm. The plate 210 comprises a thermoregulating device 226 in the form of a heating coil arranged along at least a portion of the perimeter of the first surface of the plate 210. FIG. 2a further illustrates the first axis 251 and the second axis 252 elongating along the longer dimensioned edges of the plate 210, about which the two plate sections 220 may pivot to transition between the closed position and the opened position. The two plate sections 220 are further shown joining in the center of the plate 220 at the junction line 227. It is additionally embodied that the two plate sections 220 jointly forming the first surface may be releasably connected together at the junction line 227 by means of any suitable mechanism or electro-mechanism (not shown). It is further embodied that the two plate sections 220 may at least partially overlap one another (not shown) to jointly form the first surface of the plate 210. It is additionally embodied that for a case in which the dimensions of the plate 210 are significantly large and the weight of the growth medium (not shown) by the first surface of the plate 210 is considerably heavy, a support mechanism (not shown) may be arranged beneath each of the two plate sections 220 to increase the retention capacity of the plate 210. It will further be appreciated that such support mechanism may be formed of a thermo-conductive material permitting its participation in the regulation of the temperature of the plate 210 enabled by the thermoregulating device 226. FIG. 2b shows a perspective view of another configuration of the plate 210 in a closed position. The plate 210 shown in FIG. 2b comprises three plate sections 220 joined together at two junction lines 227 to form the first surface of the plate 210. FIG. 2b further depicts a border 225 and a thermoregulating device 226 arranged along the perimeter of the plate 210. FIG. 2c illustrates a perspective view of a grid 230 in a closed position. The grid 230 comprises two grid sections 240 jointly forming the second surface of the grid 230 and joining at the junction line 228. FIG. 2c shows the two grid sections 240 being formed of a meshing 241 comprising linked threads of metallic or plastic material enabling the second surface formed by the two grid sections 240 to be covered by mesh openings 245. The mesh openings 245 are shown in FIG. 2c having similar dimensions across the second surface and which allows particles of a specific granulometric dimension to pass therethrough. FIG. 2c further illustrates the third axis 254 and the fourth axis 255 elongating along the longer dimensioned edges of the grid 230, about which the two grid sections 240 may pivot to transition between the closed position and the opened position.

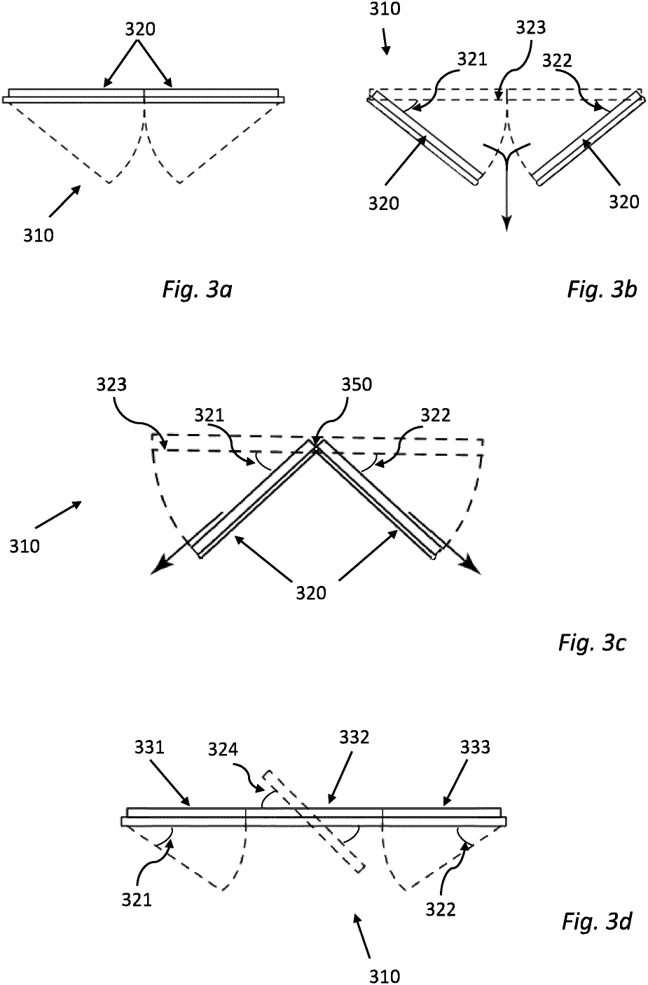

Referring now to FIG. 3a, there is shown a side view of a plate 310 in a closed position. The side view of FIG. 3a shows the smaller dimensioned edge of the plate 310 similarly dimensioned as the plate 210 of FIG. 2a. FIG. 3a depicts the plate 310 having two plate sections 320 joined at their inner edge to enable the closed position of the plate 310. The dotted lines shown in FIG. 3a illustrate the trajectory followed by the two plate sections 320 when pivoting to the opened position. FIG. 3b illustrates a side view of the plate 310 in an opened position, i.e. after pivoting of the two plate sections 320. The two plate sections 320 shown in FIG. 3b form respective angles 321, 322 with the plane 323 formed by the first surface of plate 310 in a closed position shown here in dotted lines. The respective angles 321, 322 formed by each plate sections 320 in the opened position may preferably range from 0.5° to 89.5°. IT will additionally be embodied that the respective angles 321, 322 may have different values. The plate 310 shown in the opened position in FIG. 3b permits the growth medium (not shown) to be slidably released as depicted by the arrow. FIG. 3c illustrates a side view of the plate 310 in a differently configured opened position. FIG. 3c shows the two plate sections 320 pivoted about the co-linear first axis and second axis 350 and forming respective angles 321, 322 with the plane 323. The respective angles 321, 322 formed by each plate sections 320 in the opened position may preferably range from 0.5° to 89.5°. It will additionally be embodied that the respective angles 321, 322 may have different values. The plate 310 shown in the opened position in FIG. 3c permits the growth medium (not shown) to be slidably released as depicted by the arrows. FIG. 3d illustrates a side view of the plate 310 in a closed position having three plate sections 331, 332, 333. FIG. 3d depicts in dotted lines the respective pivoting trajectories of the three plate sections 331, 332, 333, wherein the pivoting trajectories of the first and third plate sections 331, 333 is shown as a downward pivoting trajectory forming the angles 321 and 322 respectively with the closed position configuration of the plate 310. The pivoting trajectory of the second plate section 332 is shown in FIG. 3d forming the angle 324 with the plate of the closed position configuration of the plate 310. It will be appreciated that the different pivoting configuration of the plate 310 illustrated in FIGS. 3a to 3d may apply similarly to the at least one grid of the apparatus for growing biomass and its at least two grid sections.

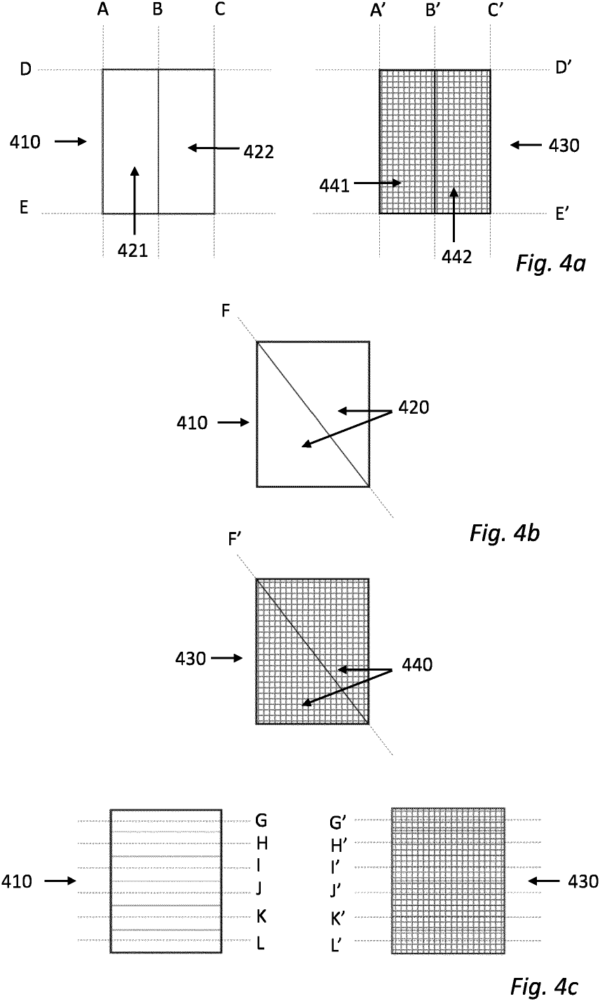

Referring now to FIG. 4a, there is shown a top view of a plate 410 in the closed position comprising a first plate section 421 and a second plate section 422 along with alternative configurations of first and second axis about which the first and second plate sections 421, 422 may pivot to transition between the closed position and the opened position. FIG. 4a shows axis A, B and E as alternatives about which the first plate section 421 may pivot, and axis C, D and E as alternatives about which the second plate section 422 may pivot. It is additionally embodied that the first and second axis may be co-linear as illustrated by axis B in FIG. 4a. Moreover, FIG. 4a illustrates a top view of a grid 430 in the closed position comprising a first grid section 441 and a second grid section 442 along with alternative configurations of third and fourth axis about which the first and second grid sections 441, 442 may pivot to transition between the closed position and the opened position. Similarly, as for the plate 410, FIG. 4a shows axis A′, B′ and E′ as alternatives about which the first grid section 441 may pivot, and axis C′, D′ and E′ as alternatives about which the second grid section 442 may pivot. It is additionally embodied that the third and fourth axis may be co-linear as illustrated by axis B′. FIG. 4b illustrates a top view of a plate 410 in the closed position comprising two plate sections 420 wherein the junction line of the two plate sections 420 is positioned along the diagonal of the plate 420. FIG. 4b illustrates the first and second axis being co-linear, i.e. axis F, and about which the two plate sections 420 may pivot to transition between the closed position and the opened position. Similarly, FIG. 4b illustrates a top view of a grid 430 in the closed position showing co-linear third and fourth axis, i.e. axis F′, about which the two grid sections 440 may pivot to transition between the closed and opened positions. FIG. 4c illustrates a top view of a plate 410 in the closed position comprising a plurality of plate sections. The plate sections illustrated in FIG. 4c each comprise co-linear first and second axis, i.e. axis G, H, I, J, K, L about which each plate section of the plate 410 may pivot to transition between the closed position and the opened position. FIG. 4c similarly illustrates a top view a top view of a grid 410 in the closed position comprising a plurality of grid sections being pivotable about co-linear third and fourth axis G′, H′, I′, J′, K′, L′ to transition between the opened and the closed position.

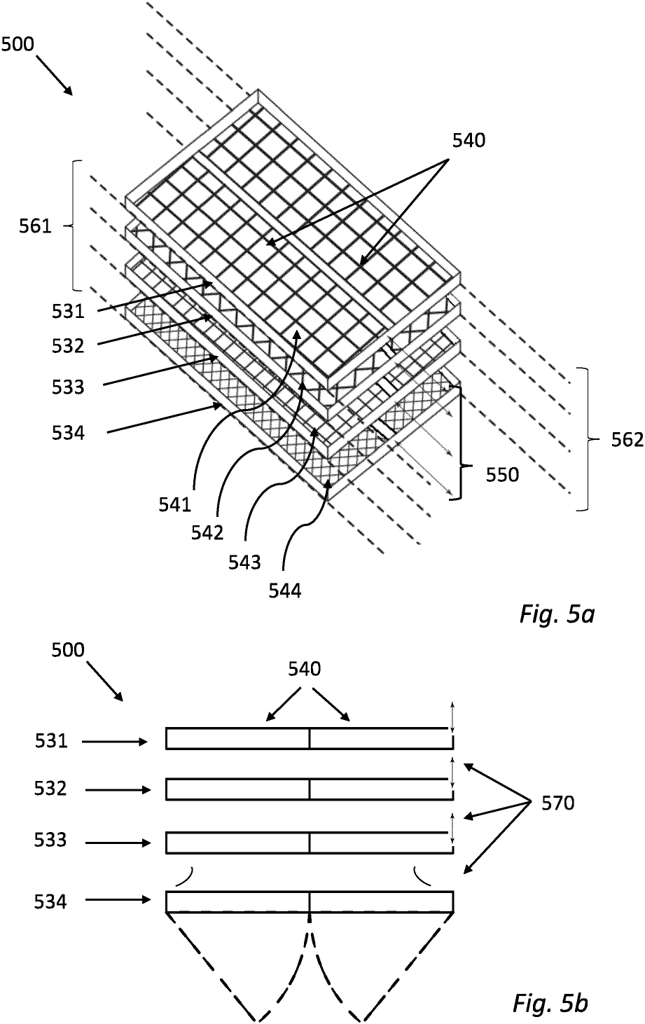

Referring now to FIG. 5a, there is shown a perspective view of a multi-layered grid assembly 500. The multi-layered grid assembly 500 is shown comprising a plurality of grids, i.e. a first grid 531, a second grid 532, a third grid 533 and a third grid 534 elevated from one another. Each grid 531, 532, 533, 534 is shown in FIG. 5a comprising two grid sections 540 jointly forming the second surface of each grids 531, 532, 533, 534 and formed of respective meshing 541, 542, 542, 544. FIG. 5a further illustrates the meshing 541 having the largest dimension of mesh opening and the meshing 544 having the smallest dimension of mesh openings. The mesh openings of the meshing 542 of the second grid 532 are shown having smaller dimensions than the mesh openings of the meshing 541 of the first grid 531, but larger than the dimensions of the mesh openings of the meshing 543 of the third grid 533. Similarly, the mesh openings of the meshing 543 of the third grid 533 are shown in FIG. 5a having larger dimensions than the mesh openings of the meshing 544 of the fourth grid 534. FIG. 5a therefore illustrates a gradual diminution of dimensions of mesh openings from the first grid 531 to the fourth grid 534 of the multi-layered grid assembly 500. Additionally, FIG. 5a depicts the third axis’ 561 and the fourth axis’ 562 elongating along the longer edges of the respective grids 531, 532, 533, 534 about which the two grid sections 540 of each grid 531, 532, 533, 534 may pivot to transition between the closed position and the opened position. Moreover, FIG. 5a shows the direction 550 of the independent movement of each grid 531, 532, 533, 534 enabled by the second drive mechanism (not shown). FIG. 5b illustrates a side view the multi-layer grid assembly 500 in which each grid 531, 532, 533, 534 is shown in a closed position. The side view of FIG. 5b shows the smaller dimensioned edge each grid 531, 532, 533, 534 and their respective grid sections 540. FIG. 5b further illustrates the elevation 570 of each grid 531, 532, 533, 534 one above the other and equally distanced. The pivoting trajectory of the two grid sections of the lower-positioned grid 534 is further depicted by the dotted line. It will be additionally embodied that the grids 531, 532, 533, 534 have similar dimensions.

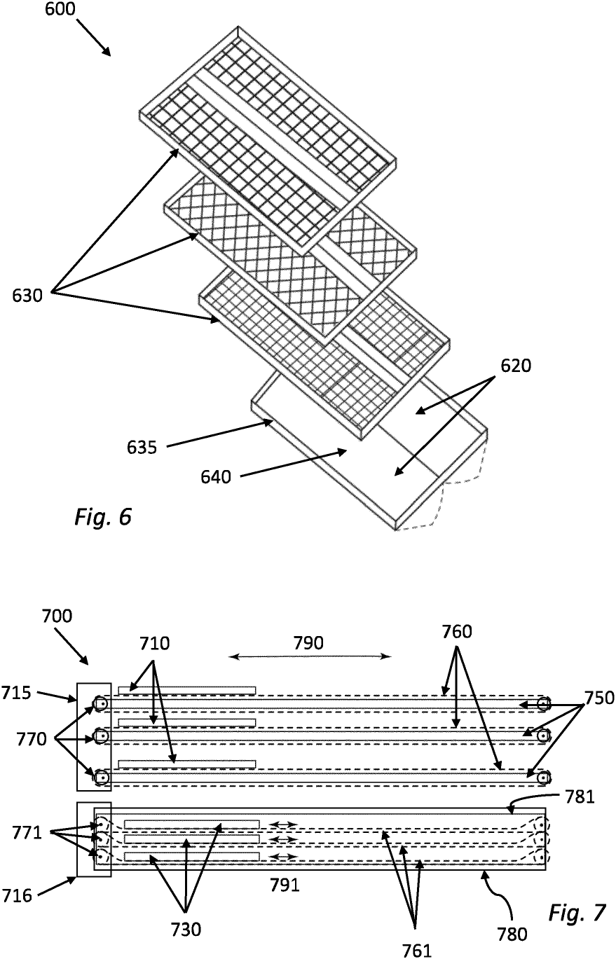

Referring now to FIG. 6, there is shown an exploded view of a multi-layer grid assembly 600 comprising three grids 630 each comprising two grid sections and respective meshing. Similarly characterized as for the multi-layered grid assembly 500 of FIG. 5a, the dimensions of mesh openings of the meshing of each grid 630 gradually reduces from the uppermost elevated grid to the lowest grid. FIG. 6 further illustrates a container 635 in the closed position and comprising two container sections 620 jointly forming a third surface 640. The two container sections 620 may pivotable between an open position and a closed position, as depicted by the pivoting trajectory shown in dotted lines in FIG. 6. It is additionally embodied that the container 635 may comprise dimensions similar to the dimensions of the three grids 630 of the multi-layer grid assembly 600.

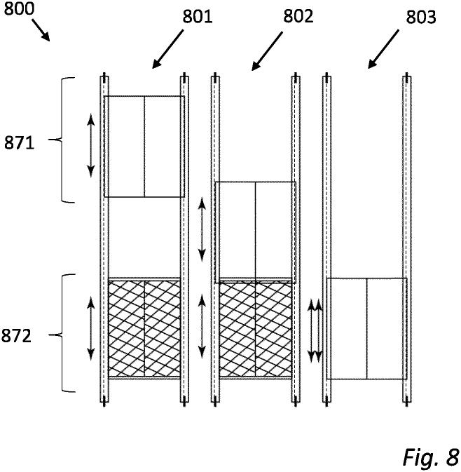

Referring now to FIG. 7, there is shown a side view of an alternative configuration of apparatus 700. FIG. 7 shows the apparatus 700 comprising a plurality of plates 710 movably supported on respective first railings 750 wherein each of the first railings 750 is positioned one above the other and above the second railing 780. The movement 790 of the plurality of plates 710 is shown in FIG. 7 enabled by the first drive mechanism 715 which comprises a plurality of chains 760 engaging the longer dimensioned edge of each plate 710 and arranged around each respective first railings 750 supporting each plate 710. FIG. 7 further depicts the first drive mechanism 715 comprising motor driven sprockets 770 for moving each chain 760 individually along each respective first railing 750, enabling the movement 790 of each plate 710. FIG. 7 further shows the second railing 780 aligned below the plurality of first railings 750 and comprising a separate channel 781 for supporting each grid 730. Similarly to the first drive mechanism 715, the second drive mechanism 716 is shown comprising a plurality of chains 761 engaging the longer dimensioned edge of each grid 730. FIG. 7 further shows the second drive mechanism comprising motor driven sprockets 771 for moving each chain 761 individually along the separate channel, enabling the movement 791 of each grid 730. It will be additionally embodied that the plurality of grids 730 form a multi-layer grid assembly.

Referring now to FIG. 8 there is shown a top view of an arrangement of apparatus for growing biomass. The arrangement 800 is illustrated in FIG. 8 comprises three apparatus 801, 802, 803 comprising plates, grids, first and second railing, first and second drive mechanism subjected to similar characterization as the apparatus described in previous FIGS. . The plate of the first apparatus 801 is shown positioned at the loading position 871 and the grid of the first apparatus 801 is shown at the discharging position. Moreover, the plate of the second apparatus 802 is show positioned between the loading position 871 and the discharging position 872 and the plate of the third apparatus is shown positioned at the discharging position 872 superimposed above the grid of the third apparatus 803. Although shown constantly at the discharging position in FIG. 8, it is embodied that the grids of each apparatus 801, 802, 803 of the arrangement of apparatus 800 are movable between the loading position 871 and the discharging position 872.

As is readily appreciated by the person skilled in the art, many modifications and variations may be devised given the above description of the principles of the inventive concept. It is intended that all such modifications and variations be considered as within the scope of the inventive concept, as it is defined in the appended patent claims.

1-15. (canceled)

16. An apparatus for growing biomass, wherein the apparatus comprises:
at least one plate comprising at least two plate sections configured to be movable between an opened position and a closed position,
wherein in the closed position the at least two plate sections jointly form a first surface for receiving and holding a growth medium for growing biomass and wherein in the opened position the at least two plate sections are pivoted away from the closed position such that the growth medium is slidably released from the at least one plate;
wherein the at least one plate is movably supported on at least one first railing, wherein the apparatus further comprises a first drive mechanism for independently moving each of the at least one plate along the at least one first railing; and
wherein the at least two plate sections are pivotable about a first axis and a second axis respectively.

17. The apparatus according to claim 16, wherein the at least one plate further comprises borders arranged along at least a portion of a perimeter of the first surface.

18. The apparatus according to claim 16, wherein the first axis and the second axis are colinear.

19. The apparatus according to claim 16, wherein one or more of said at least one plate further comprises a respective thermoregulating device arranged along at least a portion of the perimeter of the first surface, wherein the thermoregulating device is one of a refrigerant conduit, a cooling coil, a water pipe, and a heating coil.

20. The apparatus according to claim 16, wherein the first drive mechanism comprises at least one chain adapted to engage at least one edge of the at least one plate, and wherein the first drive mechanism further comprises motor driven sprockets for moving the at least one chain back and forth along the at least one first railing.

21. The apparatus according to claim 16, wherein the at least one plate is attached to the at least one first railing via at least one third railing oriented perpendicular to the at least one first railing such that the at least one plate and the at least one third railing are movable together along the at least one first railing, wherein the at least one plate is movably supported on the at least one third railing for movement along the at least one third railing, and wherein the apparatus further comprises a third drive mechanism for moving the at least one plate along the at least one third railing.

22. The apparatus according to claim 16, comprising a plurality of plates movably supported on respective first railings and movable by the first drive mechanism wherein each of the first railings are configured to be positioned one above the other and above the at least one second railing.

23. A device for separating a growth medium, wherein the device comprises:
at least one grid comprising at least two grid sections movable between an opened position and a closed position,
wherein in the closed position the at least two grid sections jointly form a second surface,
wherein said at least two grid sections comprise a meshing having a plurality of mesh openings for receiving and separating the growth medium,
wherein in the opened position the at least two grid sections are pivoted about a third and a fourth axis respectively and away from the closed position such that separated growth medium falls off the at least one grid, and
wherein the at least one grid is movably supported on at least one second railing; and
a second drive mechanism for independently moving each of the at least one grid along the at least one second railing.

24. The device according to claim 23, wherein the device comprises a plurality of said at least one grid wherein the grids are positioned elevated from one another to form part of a multi-layered grid assembly.

25. The device according to claim 23, wherein each of the grids of the multi-layered grid assembly are provided with differently sized mesh openings.

26. The device according to claim 23, wherein the at least one second railing comprises a separate channel for support of each grid, and wherein the second drive mechanism is configured to move each grid independently along the separate channel.

27. The device according to claim 23, wherein the multi-layered grid assembly comprises at its bottom a container comprising at least two container sections pivotable between an opened position and a closed position, wherein in the closed position the at least two container sections jointly form a third surface receiving and holding the separated growth medium, wherein in the opened position the at least two container sections are pivoted away from the closed position such that separated growth medium is slidably released from the container.

28. The device according to claim 23, wherein the second drive mechanism comprises at least one chain adapted to engage at least one edge of the at least one grid, wherein the second drive mechanism comprises motor driven sprockets for moving the at least one chain back and forth along the at least one second railing.

29. The device according to claim 23, wherein the at least one grid is attached to the at least one second railing via at least one fourth railing oriented perpendicular to the at least one second railing such that the at least one grid and the at least one fourth railing are movable along the at least one second railing, wherein the at least one grid is movably supported on the at least one fourth railing for movement along the at least one fourth railing, and wherein the apparatus further comprises a fourth drive mechanism for moving the at least one grid along the at least one fourth railing.

30. An arrangement comprising the apparatus according to claim 16 and a device for separating a growth medium, wherein the device comprises:
at least one grid comprising at least two grid sections movable between an opened position and a closed position,
wherein in the closed position the at least two grid sections jointly form a second surface,
wherein said at least two grid sections comprise a meshing having a plurality of mesh openings for receiving and separating the growth medium,
wherein in the opened position the at least two grid sections are pivoted about a third and a fourth axis respectively and away from the closed position such that separated growth medium falls off the at least one grid, and
wherein the at least one grid is movably supported on at least one second railing; and
a second drive mechanism for independently moving each of the at least one grid along the at least one second railing,
wherein the at least one first railing is positioned above the at least one second railing such that the at least one plate and the at least one grid are movable to a discharging position in which the at least one plate and the at least one grid are superimposed.
